(19) United States
(12) Patent Application Publication
(10) Pub. No.: US 2006/0134089 A1
(43) Pub. Date: Jun. 22, 2006

(54) Nitric oxide synthase gene transfer to the atrium for vagal tone increase

(76) Inventor: David James Paterson, Oxford (GB)

Correspondence Address: PEARL COHEN ZEDEK, LLP, 1500 BROADWAY 12TH FLOOR, NEW YORK, NY, 10036, US

(21) Appl. No.: 10/534,888

(22) PCT Filed: Nov. 13, 2003
(86) PCT No.: PCT/GB03/04934

§ 371 Date: Sep. 27, 2005

(30) Foreign Application Priority Data
Nov. 13, 2002 (GB) 02264638

(51) Int. Cl. A61K 48/00; A61K 38/44

(52) U.S. Cl. 424/94.4; 514/44

(57) ABSTRACT

High vagal tone is a positive prognostic indicator against sudden cardiac death, whereas impaired activity is a strong t predictor of mortality. NOS gene transfer to cardiac tissue has been found to increase expression of the enzyme in cholinergic ganglia, to increase the release of acetylcholine, and to enhance the heart rate response to vagal nerve stimulation. The invention provides methods and products for increasing cardiac vagal responsiveness and vagal tone and for decreasing sympathetic activity, e.g. for increasing bradycardia, for reducing cardiac autonomic impairment, for reducing the risk of sudden cardiac death, for reducing arrhythmia, for reducing the risk of myocardial infarction, and/or for reducing hypertension. The invention is particularly useful for patient groups where exercise training may be poorly tolerated. A typical method involves the step of delivering to a patient's cardiac autonomic structures nucleic acid which, when expressed, increases nitric oxide synthase levels.

All documents cited herein are incorporated by reference in their entirety.

### TECHNICAL FIELD

This invention is in the field of cardiac function, and in particular it relates to the use of gene therapy to improve cardiac vagal function and minimise excessive sympathetic stimulation.

### BACKGROUND ART

High vagal tone and responsiveness is a positive prognostic indicator against sudden cardiac death, whereas impaired activity is a strong independent predictor of mortality [1].

It is an object of the invention to provide methods and products suitable for increasing vagal responsiveness and vagal tone, and decreasing sympathetic activity.

### DISCLOSURE OF THE INVENTION

Nitric oxide (NO) generated from nitric oxide synthase (NOS) in intrinsic cardiac ganglia has been implicated in parasympathetic-induced bradycardia, and the invention is based on the finding that NOS acts in a site-specific manner to promote vagal neurotransmission and bradycardia. NOS gene transfer to cardiac tissue has surprisingly been found to increase expression of the enzyme and its immunolocalisation in cholinergic ganglia, to increase the release of acetylcholine in vitro and in vivo, and to enhance the heart rate response to vagal nerve stimulation in vitro and in vivo.

The invention therefore provides a method of treating a patient, comprising the step of delivering to the patient's cardiac autonomic structures nucleic acid which, when expressed, increases nitric oxide synthase levels. The invention also provides the use of nucleic acid which, when expressed, increases nitric oxide synthase levels, in the manufacture of a medicament.

The methods and uses of the invention are useful for increasing cardiac vagal tone and/or responsiveness, for increasing bradycardia, for reducing cardiac autonomic impairment, for reducing the risk of sudden cardiac death, for reducing arrhythmia (e.g. atrial fibrillation and/or ventricular arrhythmia), for reducing the risk of myocardial infarction, and/or for reducing hypertension [2, 3, 4]. They are particularly useful for treating patient groups where exercise training may be poorly tolerated.

Nitric Oxide Synthase

NO is formed by NOS from L-arginine, and the reaction requires tetrahydrobiopterin (BH₄) as a cofactor. It is thought to be a fundamental signalling molecule in the regulation of cardiac cholinergic function [e.g. see refs. 5 to 8], and neuronal nitric oxide synthase (NOS-1 or nNOS) co-localizes with choline acetyltransferase in the intra-cardiac ganglia [9]. Functionally, pharmacological evidence suggests that NO generated from NOS-1 directly enhances the negative chronotropic effect of cholinergic stimulation [10,11] by activating the guanylate cyclase/cGMP pathway [11,12] to facilitate the release of acetylcholine (ACh) [13], and indirectly via endothelial NOS-3 (eNOS) M₂ receptor coupled inhibition of I_{Ca-L} in pacemaking cells [14], although this latter point is disputed [15]. The vagal heart rate response to modulators of the NO-cGMP pathway is not mimicked by carbachol (a stable analog of acetylcholine), suggesting that the dominant functional role of this pathway is pre-synaptic to the neuro-effector junction [11,12].

The invention will typically involve delivering nucleic acid encoding a nitric oxide synthase to a patient, with increased levels of NOS leading to increased levels of NO and/or superoxide. Three types of nitric oxide synthases are recognised: NOS-1 (or nNOS), NOS-2 (iNOS) and NOS-3 (eNOS). All three enzymes have been cloned and sequenced for various organisms, and polymorphic variants, splice variants and isoforms of NOS genes have been described [e.g. see refs. 16 to 19, etc.]. NOS-1 and NOS-3 are constitutively expressed in vivo (collectively referred to as ‘cNOS’) whereas expression of NOS-2 is inducible. In general, the term “nitric oxide synthase” encompasses all three NOS forms, and includes both natural and modified forms of the enzyme (e.g. those obtained by protein engineering, mutagenesis, molecular evolution, fusion proteins, etc.) provided that the enzyme retains the ability to convert L-Arginine to nitric oxide (e.g. as specified by enzyme classification EC 1.14.13.39) and/or the ability to produce superoxide.

The invention preferably uses a cNOS (i.e. a NOS-3 and/or a NOS-1), with NOS-1 being more preferred. Detailed information about NOS-1 is available from reference 20.

The NOS is preferably from the organism being treated e.g. human NOS is used to treat humans.

The Nucleic Acid

The invention involves delivery of nucleic acid encoding NOS to the patient's cardiac tissue. As mentioned above, detailed sequence information is available for all three forms of NOS, and the use of all three forms in gene therapy has been described [e.g. as reviewed in ref. 21].

The nucleic acid will generally take the form of DNA.

The nucleic acid may be replicating or non-replicating.

The nucleic acid may be integrating or non-integrating.

The nucleic acid may be an autonomously replicating episomal or extrachromosomal vector, such as a plasmid. Eukaryotic expression vectors for NOS are disclosed e.g. in ref. 22.

The nucleic acid preferably includes at least one transcriptional promoter and/or enhancer for controlling expression of the NOS sequence. Typical nucleic acids include an upstream promoter for controlling transcription of the NOS coding sequence. Transcriptional repressors may also be used.

The promoter may be a constitutive promoter or it may be a regulated or inducible promoter. The promoter will typically not be the promoter with which the NOS coding sequence is associated in nature i.e. the nucleic acid is a heterologous construct. The promoter may be naturally-occurring, but will generally be a chimeric regulatable system incorporating various prokaryotic and/or eukaryotic elements. Various promoter modules can be used to allow various levels of control.

Constitutive promoters useful for directing transcription of the NOS coding sequence include those from genes coding for glycolytic enzymes, or from β-actin, and allow persistent up-regulation of NOS expression. Viral promoters may also be used e.g. from CMV (as in refs. 31 & 33, with nNOS).

Inducible and regulated promoters [e.g. ref. 23] can allow spatial and/or temporal control of NOS transcription, above and beyond any spatial control achieved by targeted delivery of the vector.

Tissue-specific or cell-type-specific promoters facilitate spatial control. Advantageously, the invention uses a promoter which is active in neuronal tissue, particularly tissue in the autonomic nervous system (e.g. the vagus). Preferred promoters are active in cholinergic ganglia tissue e.g. the promoter from choline acetyltransferase or from the vesicular acetylcholine transporter.

Drug-inducible promoters facilitate temporal control e.g. by including cAMP response element enhancers in a promoter, cAMP modulating drugs can be used [24]. Other common regulated systems are based on tetracycline, RU486, ecdysone, rapamycin, etc. [23]. In contrast, repressor elements can be included in a vector to prevent transcription in a drug's presence [25]. Spatial and temporal control of gene expression can also be achieved by using a promoter which responds to ionising radiation [e.g. refs. 26 & 27].

As mentioned above, these various promoters can be used alone or in combination to allow multiple control mechanisms. In addition to the promoter, the nucleic acid may include transcriptional regulatory sequences (e.g. enhancers, upstream and/or downstream) to interact functionally with the promoter. Nucleic acid can also include a eukaryotic transcriptional terminator sequence downstream of the NOS coding sequence.

Other features of nucleic acids of the invention include: a signal which directs polyadenylation of NOS-coding RNAs (e.g. from SV40); a selectable marker; an origin of replication; a multiple cloning site; and an IRES. For non-replicating nucleic acids, the origin of replication is preferably active in prokaryotes but not eukaryotes, thereby facilitating production in convenient prokaryotic systems.

The invention therefore provides a nucleic acid molecule comprising a non-constitutive promoter and a coding sequence, wherein: (a) the promoter is operably linked to the coding sequence to control transcription of the coding sequence; (b) the promoter is specifically active in cholinergic ganglia tissue; and (c) the coding sequence encodes a nitric oxide synthase. Further features of the nucleic acid are as described above. The invention also provides this nucleic acid for use as a medicament.

Delivering the Nucleic Acid

For delivery to a patient, nucleic acid of the invention may be administered as “naked” nucleic acid. More typically, however, it will be packaged for delivery within a vector suitable for use in gene therapy [28].

The invention can utilise viral vectors [29,30] (e.g. adenovirus vectors [31,32,33], adeno-associated virus vectors [34], lentivirus vectors [35], parvovirus vectors, herpesvirus-based vectors, other retroviral vectors, alphavirus vectors, etc.), but non-viral vectors [36,37,38] are preferred. Suitable delivery systems for vectors include liposomes (e.g. cationic or anionic [39 to 43]), microparticles, nanoparticles, condensing agents, bolasomes, lipoplexes, virosomes, cationic polymers, etc. Nucleic acids of the invention may therefore include suitable packaging signals.

Viral and non-viral vectors can be administered to a patient in various ways e.g. by hand-held gene transfer particle gun [44], or by injection of the vector.

Delivery of the vector may be systemic, but will generally be targeted to the heart, to the autonomic nervous system, or specifically to the vagus nerve. Targeting may be receptor-mediated (e.g. using immunoliposomes), as described in, for example, references 45 to 50. As an alternative, targeting may involve direct delivery to target tissue (e.g. by direct injection of nucleic acid into the vagus nerve, which may be convenient during a procedure such as open heart surgery, or by injection into the heart in the vicinity of the cardiac/vagus interface).

Another targeted delivery method (particularly for non-viral vectors) involves administering the vector to the body and “activating” it specifically in the heart. For instance, references 51 & 52 review the use of microbubbles which contain gene therapy vectors, with ultrasound being used to disrupt the microbubbles in a site-specific manner to deliver nucleic acid. Reference 53 describes a method where vector delivery is promoted by echo contrast microbubble injection into the aortic root with brief balloon occlusion above the sinuses.

Therapeutic compositions containing a nucleic acid are typically administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. Factors such as method of action and efficacy of transformation and expression are considerations which will affect the dosage required for ultimate efficacy. Where greater expression is desired over a larger area of tissue, larger amounts of vector or the same amounts re-administered in a successive protocol of administrations, or several administrations to different adjacent or close tissue portions may be required to effect a positive therapeutic outcome. In all cases, routine experimentation in clinical trials will determine specific ranges for optimal therapeutic effect.

Delivery of nucleic acid can occur in vivo or ex vivo. Ex vivo gene therapy requires the isolation and purification of patient heart cells, the introduction of a therapeutic gene and introduction of the genetically altered cells back into the patient. In contrast, in vivo gene therapy does not require isolation and purification of a patient's cells.

The Cardiac Tissue

The invention involves the delivery of nucleic acid to cardiac tissue. More particularly, it involves delivery to the autonomic nervous system of the heart ie. to the vagus nerve. Expression of the nucleic acid in the receiving tissue increases nitric oxide synthase levels there.

For most delivery methods, other tissues and cell-types will inevitably receive the vector as well. This “bystander” transfection may be acceptable, and may actually be advantageous e.g. increased NO levels in neurons of the sympathetic nervous system can be inhibitory, thereby reinforcing the effect of vagal stimulation. Therefore delivery need not be vagus-specific. If necessary, a high degree of control can be achieved by using both targeted delivery of the NOS-coding sequence and target-specific control of transcription.

If a “homing” vector is used (e.g. a receptor-mediated vector) then the vector can be administered to a patient remotely from the heart (e.g. by intravenous injection). Remote administration can also be used where site-specific activation of transfection is used (e.g. ultrasound activation). Other vectors will, however, generally require direct delivery to the heart (e.g. by direct injection, transcoronary delivery, percutaneous injection, etc.).

Within the heart, delivery to the right atrium is preferred e.g. injection into the right atrium where the vagal post-ganglionic cell bodies terminate, close to their neuroeffector junction.

The Increase in Vagal Tone

Vagal tone is the effect produced on the heart when only the parasympathetic nerve fibres (carried in the vagus nerve) are controlling the heart rate, and reflects the extent of restraint on the heart applied by the brain. High cardiac vagal tone is a positive prognostic indicator against sudden cardiac death, and diminished vagal tone is a known mortality marker. A reduction of vagal tone is also found to be associated with acute myocardial infarction. The invention aims to increase vagal tone, thereby reducing the risk of cardiac death. Vagal tone can be increased by aerobic exercise but, for patients where such training may not be tolerated, the invention offers an alternative.

Various methods are known for measuring vagal tone. It can be monitored in a non-invasive, real time and continuous manner using apparatus such as the NeuroScope (MediFit Diagnostics Ltd, London) [e.g. refs. 54 & 55]. A linear measurement scale has been devised [56] called the linear vagal scale (LVS).

The method of the invention preferably increases vagal tone, as measured by LVS, by at least 10% (e.g. an increase of at least 20%, 30%, etc.) relative to the value prior to commencement of treatment.

The effect of increased vagal tone can also be seen in bradycardia i.e. in a reduction of heart rate. The method of the invention preferably reduces the heart rate of a patient by at least 10 beats per minute (e.g. by at least 20 bpm, 30 bpm, 40 bpm, 50 bpm or more) e.g. to fewer than 60 beats per minute.

Overall, the effect of the invention is to increase cardiac vagal tone in a way which mimics the effect of athletic/aerobic exercise.

The Patient

The patient will usually be a mammal, such as a horse (e.g. a racehorse), a dog (e.g. greyhound), a cow, etc. Preferred patients are humans, and generally adult humans. The patient will generally have a low cardiac vagal tone.

Medicaments

The invention provides a medicament comprising nucleic acid encoding nitric oxide synthase as described above. The nucleic acid (either naked or in the form of a gene therapy vector) will generally be combined with a pharmaceutically acceptable carrier.

The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles. Typically, the therapeutic compositions are prepared as injectables, either as liquid solutions or suspensions; solid forms suitable for solution in, or suspension in, liquid vehicles prior to injection can also be prepared. Pharmaceutically acceptable salts can also be present in the pharmaceutical composition, e.g. mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in reference 57.

Preferred medicaments are aqueous, buffered at pH 7.0±0.5, pyrogen-free and sterile.

Medicaments of the invention may be administered in combination with beta-blockers, thereby achieving a decrease in sympathetic nervous activity at the same time as increasing vagal parasympathetic activity.

### DEFINITIONS

The term “comprising” means “including” as well as “consisting” e.g. a composition “comprising” X may consist exclusively of X or may include something additional e.g. X+Y.

The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

### BRIEF DESCRIPTION OF DRAWINGS

FIG. 1 shows NADPH-diaphorase staining of atrial cryosections. The bar is 25 μM.

FIG. 2 shows anti-GFP histochemical staining of atrial cryosections. The bar is 50 μM.

FIG. 3 is a western blot (17.5 μg protein loaded) of protein extracts from atria transfected with eGFP or with NOS-1.

FIG. 4 shows the same data quantitatively.

FIG. 5 shows in vivo heart rate responses to vagal nerve stimulation (5 Hz).

FIG. 6 is a graph of the same response data (*p<0.05 unpaired t-test n=6)

FIG. 7 shows heart rate responses to vagal nerve stimulation (3 Hz & 5 Hz; 30 s, 15V, 1 ms duration).

FIG. 8 shows heart rate responses to bath-applied carbachol (CCh; 2 minutes incubation).

FIG. 9 shows the release of ACh in the presence of absence of the guanylyl cyclase inhibitor 1H-(1,2,4)-oxadiazolo-(4,3-a)-quinoxalin-1-one (ODQ 10 μM). (*p<0.01 paired t test; †p<0.05 ?²).

FIG. 10 shows heart rate responses to vagal nerve stimulation in the presence of absence of the NOS inhibitor N?-nitro-L-arginine (L-NA, 100 μM, 20 min incubation).

FIG. 11 shows heart rate responses to bath-applied carbachol (0.2 μM; 2 mins incubation) in atria pre-treated with L-NA (100 μM, 20 min incubation).

FIG. 12 shows in vivo heart rate reductions in the hypertensive rats.

FIG. 13 shows in vivo noradrenaline responses in the hypertensive rats. The upper series of data shows responses from the Ad.eGFP group; the lower series of data is the Ad.NOS-1 group.

FIG. 14 shows confocal imaging of atria from Ad.eGFP group (top row) and Ad.NOS-1 group (bottom row) stained for NOS-1 (left column) and choline acetyltransferase (CHAT, middle column). NOS-1 staining in cholinergic ganglia was greatly increased in Ad.NOS-1 compared to Ad.eGFP groups. The right column shows co-localisation of NOS-1 and ChAT.

FIG. 15 shows the heart rate (bpm) of SHR rats in response to noradrenaline. Rats were transfect either with Ad.eGFP (upper series, n=20) or with Ad.NOS-1 (lower series, n=8).

FIG. 16 shows the heart rate response (bpm) of SHR rats in VNS experiments. The left-hand column of each pair is from Ad.eGFP rats and the right-hand column is from Ad.NOS-1 rats.

FIG. 17 shows the heart rate (bpm) of SHR rats (upper series, n=20) and WKY rats (lower series, n=6) in response to noradrenaline. All rats were transfect with Ad.eGFP.

### MODES FOR CARRYING OUT THE INVENTION

Aerobic Training Up-Regulates the NO-Mediated Signalling Pathway in the Heart

Groups of mice were either trained by giving them access to a running wheel for a period of 10 weeks (+EX) or housed in cages without wheels for the same period (−EX). Cardiac ganglia were then visualised by confocal microscopy after treatment of atrial tissue for immunohistochemistry.

Around 19% of cholinergic neurones in intrinsic parasympathetic ganglia exhibited co-immunoreactivity for NOS-1 following training. Furthermore, western blot analysis for NOS-1 expression in homogenised atria revealed a 75% increase in expression in the trained group.

To test the functional action of the vagus nerve on heart rates in the two groups, atria were isolated with an intact vagus attached to a stimulator. Atria from trained mice demonstrated enhanced bradycardia with vagal nerve stimulation compared to the controls (+EX: −76±8 bpm (3 Hz), −109±13 bpm (5 Hz); −EX: 62±7 bpm (3 Hz), −93+9 bpm (5 Hz); p<0.01).

Responses to carbamylcholine (CCh), the acetycholine analog, gave equivalent bradycardia in both groups, suggesting that training induces presynaptic rather than postsynaptic facilitation of parasympathetic function.

Inhibition of NOS-1 with L-NVIO, a specific pharmacological agent, decreased the bradycardia associated with nerve stimulation in both groups. The effect was more pronounced in the +EX group, however, and normalised the response with respect to the control, leading to the conclusion that the peripheral effects of exercise are mediated principally by the elevated NOS-1 levels seen after training. The effect of L-VNIO was reversed by addition of excess L-arginine, the substrate for NOS-1.

Gene Transfer into Heart Tissue

NOS-1 gene transfer into hearts was tested as a route for reproducing the trained vagal phenotype. Guinea pigs were treated with recombinant adenoviruses containing genes for NOS-1 (Ad.NOS-1) or enhanced green fluorescent protein (Ad.eGFP), or with PBS alone (Sham) into the right atrium.

Replication-deficient adenoviral vectors were generated, purified and delivered as described in reference 58. Percutaneous gene transfer to the atrial wall of 5×10⁹-5×10¹⁰ virus particles of Ad.eGFP (n=34) or Ad.NOS-1 (n=33) in 300 μl phosphate-buffered saline (PBS) or sham injections (n=9, PBS only) was performed in halothane-anaesthetised male guinea pigs (mean wt, 374±5 g).

Following ˜5 days vector incubation, samples of atrial tissue were snap-frozen in OCT medium after equilibration in either 4% paraformaldehyde/2% sucrose or liquid nitrogen. Samples were tested by immunohistochemistry [59], confocal microscopy, NADPH-diaphorase assay, fluorescence microscopy [60], and western blot.

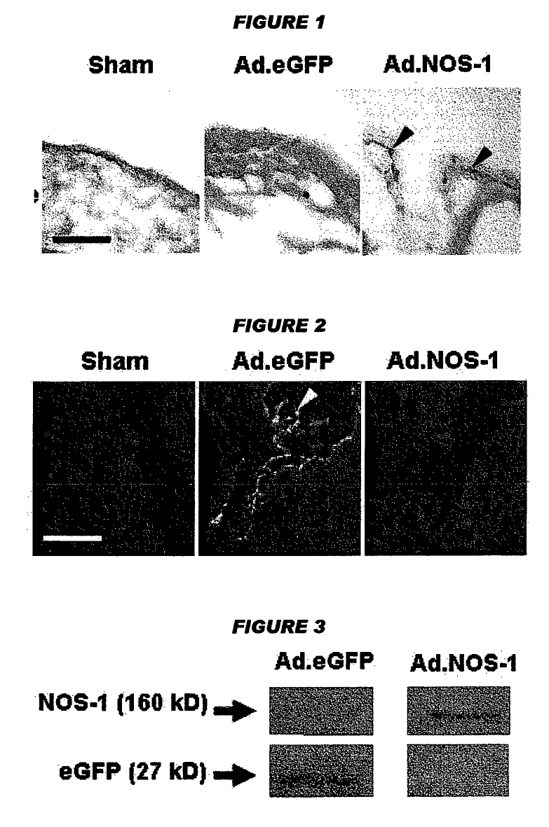

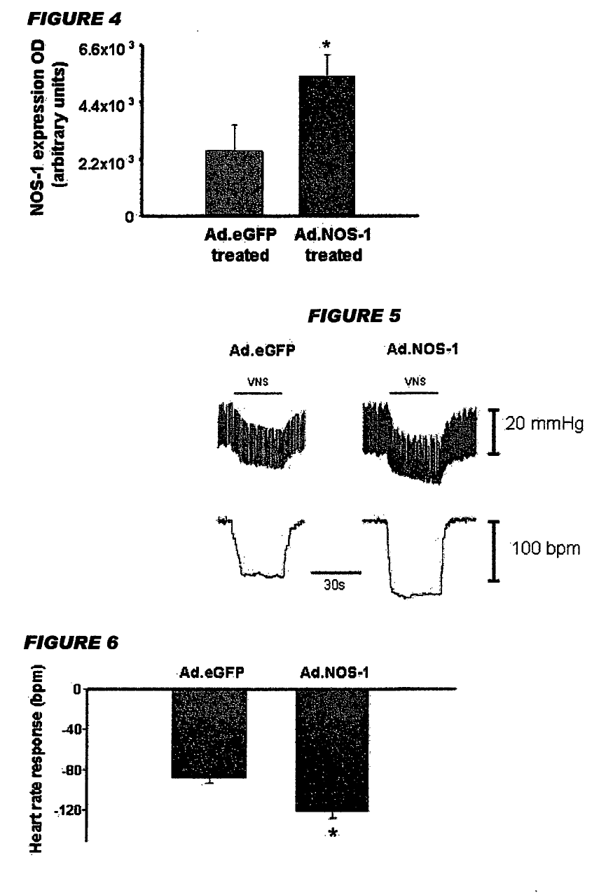

Qualitative examination of NOS location using NADPH-diaphorase staining of tissue cryosections showed greater expression in atrial tissue only in the Ad.NOS-1 group (FIG. 1). Western blotting of NOS-1 gave the same result (FIGS. 3 & 4; 110% increase in NOS-1 expression). eGFP was present only in atria from Ad.eGFP-treated animals, using both western blotting and fluorescence microscopy (FIGS. 2 & 3). Expression of β-actin was equal between all groups.

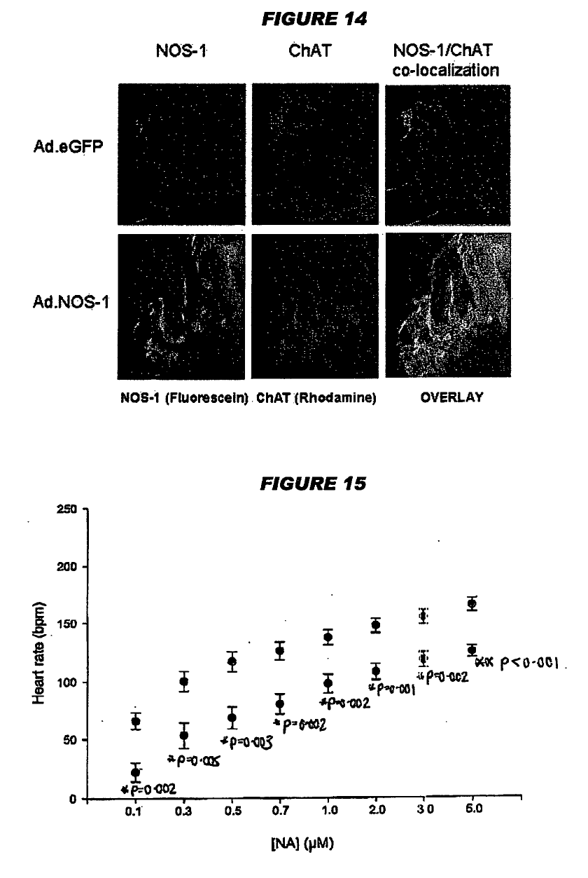

Co-localisation of NOS-1 and cholinergic ganglia was revealed by confocal microscopy using immunofluorescence in whole atria against sheep α-NOS-1 and goat α-ChAT (Chemicon, UK). These data indicate site-specific pick up of the transgene into neurons (FIG. 14).

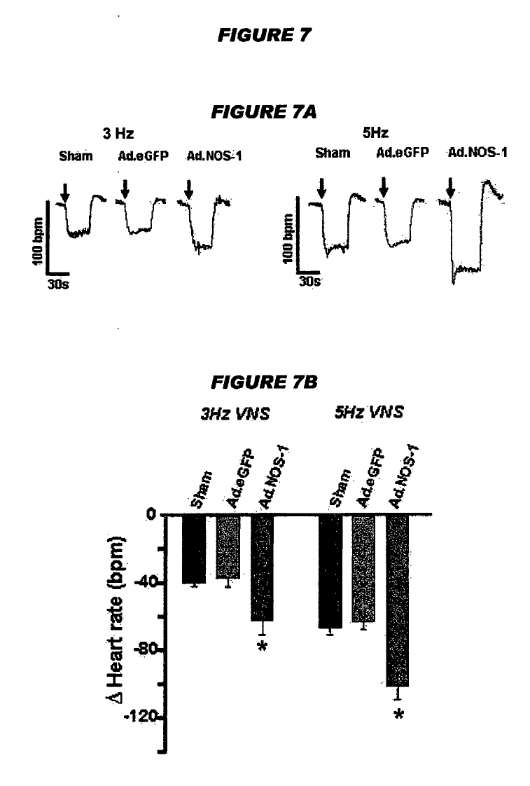

In vitro heart rate (HR) responses to vagal nerve stimulation (VNS) were measured (3 & 5 Hz; 10V, 1 ms pulse interval). As shown in FIGS. 5 to 7, HR responses to vagal stimulation were increased (p<0.01) in Ad.NOS-1 subjects (−68±10 bpm (3 hz); −105±10 bpm (5 Hz)) compared to Ad.eGFP (−32±4 bpm (3 hz); −61±6 bpm (5 Hz)). Sham responses were no different to the Ad.eGPF group (−39±3 bpm (3 hz); −67±5 bpm (5 Hz)), confirming that eGFP expression has no side-effect on NOS-1 expression.

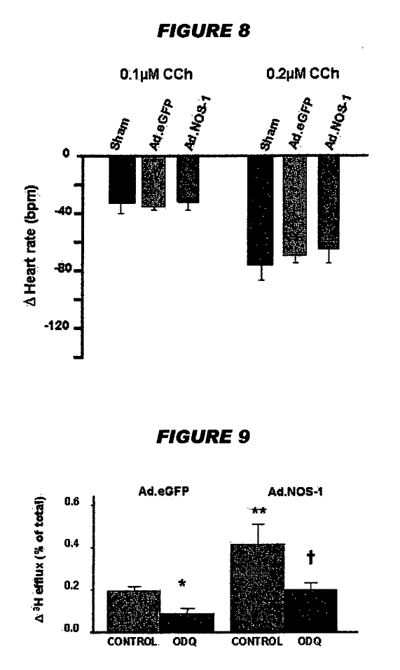

HR responses was also monitored after treatment with bath-applied carbachol (CCh, Sigma UK) in vitro (10⁻⁷ & 3×10⁻⁷ mol/L). There was no response difference between the three groups (FIG. 8).

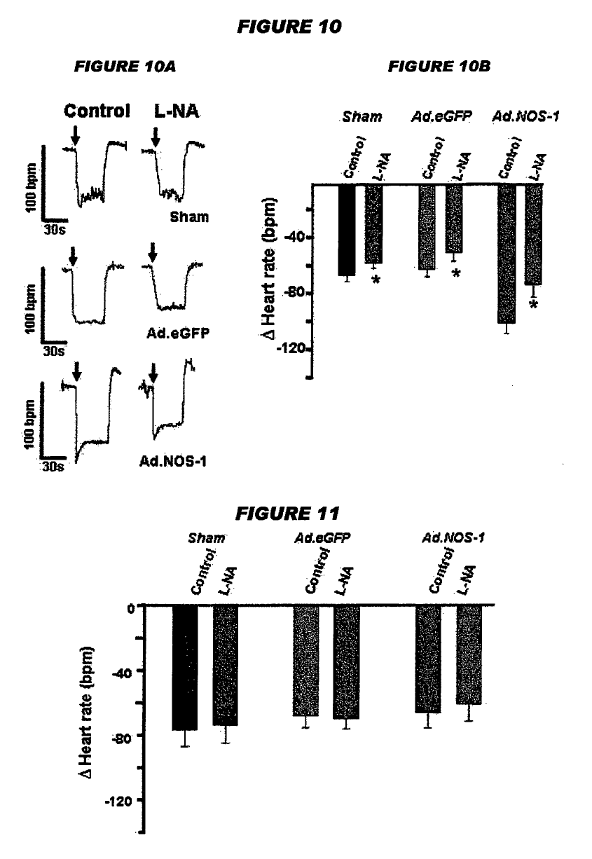

Isolated atria from all groups were pre-treated in vitro with N?-nitro-L-arginine (L-NA, Sigma UK), a NOS inhibitor. This pre-treatment significantly attenuated the enhanced vagal bradycardia in Ad.NOS-1-treated atria (*p<0.001, FIG. 10) and the HR responses of Ad.eGFP-treated (*p<0.01, n=13) and sham preparations (*p<0.01, n=5). Following NOS inhibition, responses of Ad.NOS-1, Ad.eGFP and sham-treated preparations to 3 Hz and 5 Hz VNS were not significantly different (FIG. 10). In contrast, there were no significant differences among responses of the three treated groups to bath-applied carbachol (FIG. 8). Furthermore, there was no significant difference among responses of the three groups to bath-applied carbachol in atria pre-treated with L-NA (FIG. 11), suggesting that NO generated from NOS-1 acts predominantly within intra-cardiac ganglia to enhance cholinergic regulation of cardiac function without affecting post-synaptic cholinergic signalling. These data are consistent with reports that pharmacological enhancement of the NO-cGMP pathway during right atrial field stimulation facilitates the release of acetylcholine [13] by activating the guanylate cyclase/cGMP pathway [11,12]. Moreover, the vagal HR response to pharmacological modulators of the NO-cGMP pathway is not mimicked by bath applied cholinergic analogs, confirming that the main functional response of the NO-cGMP pathway resides pre-synaptically.

Acetylcholine release was measured in vitro before and after inhibition of guanlyl cyclase, as described in ref. 13. NOS-1-treated atria had enhanced (p<0.01) release of ACh during field stimulation compared to eGFP-treated atria. This difference was abolished by inhibition of guanlyl cyclase using ODQ (FIG. 9).

Further experimental details can be found in reference 61 and its data supplement.

Rat Model of Hypertension

Following on from the in vitro guinea pig results, rats were transfected with the same Ad.NOS-1 and Ad.eGPF adenovirus vectors. The spontaneously hypertensive rat (SHR) standard model was used, and heart rate responses were measured in vivo.

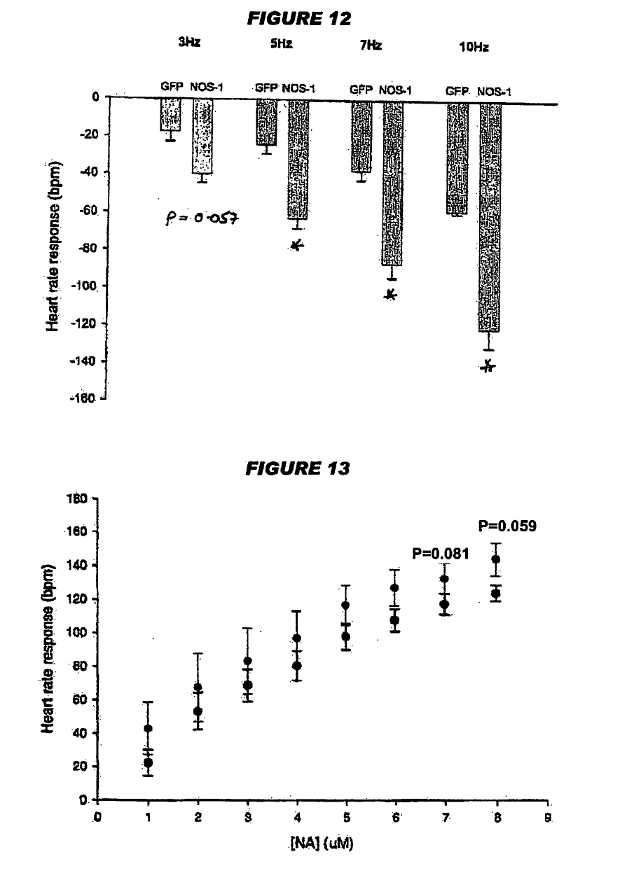

At all stimulation frequencies, the reduction in heart rate was significantly greater in the Ad.NOS-1 group than in the Ad.eGFP group (FIG. 12). Similarly, the increase in heart rate in response to injection of noradrenaline was significantly higher in the Ad.eGFP group than in the Ad.NOS-1 group FIG. 13).

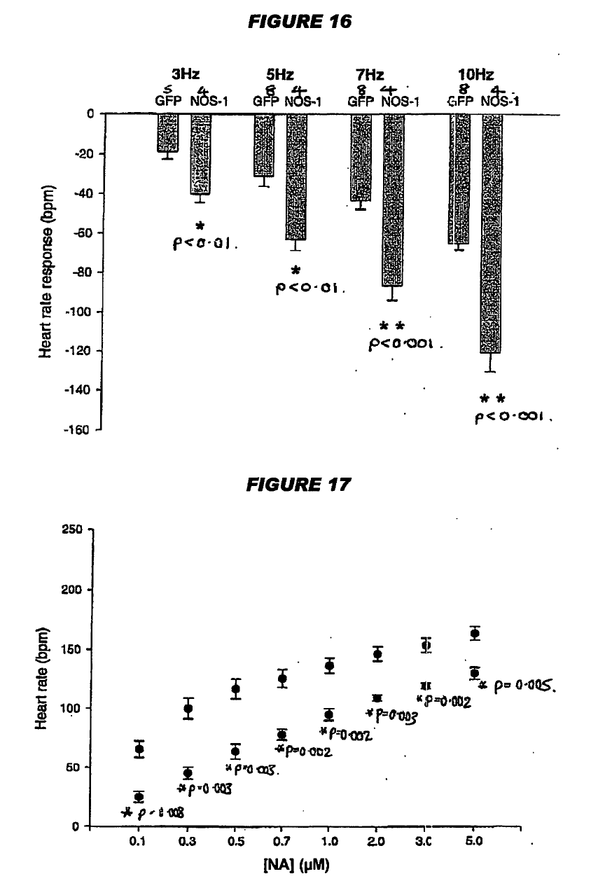

Results of a further experiment in SHR comparing in vivo noradrenaline responses in an Ad.eGPF group with a NOS-1 group are shown in FIG. 15. Results were significant (*) at all noradrenaline concentrations (p<0.005, and often p≦0.002). As well as these data on the sympathetic nervous system, parasympathetic responses to in vivo VNS were significantly different in the two groups at 3 Hz, 5 Hz, 7 Hz and 10 Hz (FIG. 16).

The standard normotensive control for SHR is the Wistar-Kyoto (WKY) outbred rat. The noradrenaline-induced reduction in heart rate achieved by expression of NOS in the SHR model is similar to the reduction seen in WKY rats compared to the SHR group. FIG. 17 shows noradrenaline responses in Ad.eGFP-transfected SHR rats and Ad.eGFP-transfected WKY rats, and the results are similar to those seen when comparing Ad.eGFP and Ad.NOS-1 groups in FIG. 15.

Cardiac transfection with NOS-1 therefore results in enhanced in vivo vagal responsiveness in the hypertensive rat, and there is a trend for decreased adrenergic responses. Beyond the in vitro guinea pig studies, therefore, these in vivo findings suggest that transfection with NOS offers a positive prognostic outcome in relation to minimising arrhythmia and sudden cardiac death.

### CONCLUSIONS

NOS-1 acts in a site-specific manner to promote vagal neurotransmission and bradycardia.

NOS-1 gene transfer to the guinea pig right atrium increases protein expression and NOS-1 immunolocalisation in cholinergic ganglia. It also increases the release of acetylcholine and enhances heart rate responses to vagal nerve stimulation in vitro and in vivo.

NOS inhibition normalises in vitro HR responses to vagal nerve stimulation in NOS-1 treated animals compared to control groups. In contrast, an acetylcholine analog reduces HR to the same extent in all groups before and during NOS inhibition. NOS-1-derived NO thus acts pre-synaptically to facilitate vagally-induced bradycardia.

The increase in vagal function following NOS-1 gene transfer is due to enhanced NO bioavailability.

Up-regulation of NOS-1 via gene transfer is useful for increasing cardiac vagal function.

It will be understood that the invention has been described by way of example only and modifications may be made whilst remaining within the scope and spirit of the invention.

### REFERENCES (THE CONTENTS OF WHICH ARE HEREBY INCORPORATED BY REFERENCE)

[1] Cole C R et al. N Engl J Med. 1999;341:1351-7.

[2] Petretta et al. (1995) Am J Hypertens. 8:1206-1213.

[3] Bibevski & Dunlap (1999) Circulation 99:2958-2963.

[4] Grassi et al. (1999) Am J Cardiol. 84:525-529.

[5] Balligand J L. Cardiovasc Res. 1999;43:607-20.

[6] Chowdhary S, Townend J N. Clin Sci (Colch). 1999;97:5-17.

[7] Paterson D J. Expt Physiol. 2001;86:1-12

[8] Wegener J W et al. Circ Res. 2002;90:18-20.

[9] Choate J K et al. Am J Physiol Heart Circ Physiol. 2001;281:H2310-7.

[10] Conlon K, Kidd C. J Auton Nerv Syst. 1999;75:136-46.

[11] Herring N et al. J Mol Cell Cardiol. 2000;32:1795-1804.

[12] Sears C E et al. J Appl Physiol. 1999;86:510-6.

[13] Herring N, Paterson D J. J Physiol. 2001;535:507-18

[14] Han X et al. J Physiol (Lond). 1994;476:309-14.

[15] Vandecasteele G et al. Nature Medicine. 1999;5:331-334.

[16] Liou Y J et al. Am J Med Genet 2002; 114:687-688.

[17] de Prado A et al. Adv Perit Dial 2002;18:18-20.

[18] Singleton A B et al. Neurosci Lett 2001;303:33-36.

[19] Brenman J E et al. Dev Neurosci 1997;19:224-231.

[20] Wang Y et al. Crit Rev Neurobiol 1999;13:21-43.

[21] Chen et al. Jpn J Pharmacol 2002 89:327-336.

[22] U.S. Pat. No. 6,146,887.

[23] Agha-Mohammadi & Lotze (2000) J. Clin. Investigation 105:1177-1183.

[24] Suzuki et al. (1996) Human Gene Therapy 7:1883-1893.

[25] Hu et al. (1997) Cancer Research 57:3339-3343.

[26] WO 92111033

[27] Hallahan et al. (1995) Nature Medicine 8: 786-791.

[28] Vector Targeting for Therapeutic Gene Delivery. Curiel & Douglas (eds.) ISBN: 0471434795.

[29] Viral Vectors for Gene Therapy: Methods and Protocols. MacHida & Constant. ISBN 1588290190.

[30] Viral Vectors: Basic Science and Gene Therapy. Cid-Arregui et al. ISBN 188129935X.

[31] West et al. (2001) Circulation 104:1526-1532.

[32] Adenoviral Vectors for Gene Therapy. Curiel & Douglas (eds.) ISBN: 0121995046.

[33] Li et al. (2002) Am J Physiol Heart Circ Physiol 282:H594-601.

[34] Adeno-Associated Virus (Aav) Vectors in Gene Therapy. Bems & Giraud. ISBN: 3540610766.

[35] Lentiviral vectors. Trono et al. (eds.) ISBN: 3540421904.

[36] Nonviral Vectors for Gene Therapy. Huang et al. ISBN: 0123584655.

[37] Nonviral Vectors for Gene Therapy: Methods and Protocols. ISBN: 0896037126.

[38] Li & Ma Curr Gene Ther 2001;201-226.

[39] U.S. Pat. No. 5,422,120

[40] WO 95/13796

[41] WO 94/23697

[42] WO 91/14445

[43] EP 0524968

[44] U.S. Pat. No. 5,149,655

[45] Findeis et al., Trends Biotechnol. (1993) 11:202

[46] Chiou et al. (1994) Gene Therapeutics: Methods And Applications Of Direct Gene Transfer. ed. Wolff

[47] Wu et al., J. Biol. Chem. (1988) 263:621

[48] Wu et al., J. Biol. Chem. (1994) 269:542

[49] Zenke et al., Proc. Natl. Acad. Sci. (USA) (1990) 87:3655

[50] Wu et al., J. Biol. Chem. (1991) 266:338

[51] Unger E C et al. Eur J Radiol 2002 42:160-168.

[52] Main & Graybum. Am Heart J 1999 137:144-153.

[53] Beeri G et al. Circulation 2002 106;1756-1759.

[54] Little et al. Am J Physiol. 1999;276:H758-765.

[55] www.medifitdiagnostics.com/html/neuro.html

[56] Julu P O. J Auton Pharmacol 1992;12:109-115.

[57] Gennaro (2000) Remington: The Science and Practice of Pharmacy. 20th edition, ISBN: 0683306472

[58] Channon et al. (1996) Cardiovasc. Res. 32:962-972.

[59] Wang & Morris (1996) Neuroscience 74:1059-1068.

[60] Channon et al. (1998) Circulation. 98:1905-1911.

[61] Mohan et al. (2002) Circ Res 91:1089-1091.

1. A method of treating a patient, comprising the step of delivering to said patient's cardiac autonomic structures a nucleic acid molecule which, when expressed, increases nitric oxide synthase levels.

2. A pharmaceutical composition comprising a nucleic acid molecule which, when expressed in cardiac autonomic structures, increases nitric oxide synthase levels.

3. The method of claim 1, wherein treatment is for increasing cardiac vagal tone, increasing cardiac vagal responsiveness, increasing bradycardia, reducing cardiac autonomic impairment, reducing the risk of sudden cardiac death, reducing arrhythmia reducing the risk of myocardial infarction or reducing hypertension.

4. The method of claim 1, wherein said nucleic acid molecule is delivered to the vagus nerve.

5. The method of claim 1, wherein said nitric oxide synthase is NOS-1 or NOS-3.

6. The method of claim 5, wherein the NOS-1 is human NOS-1.

7. The method of claim 1, wherein said nucleic acid molecule is targeted to cardiac tissue.

8. The method of claim 1, wherein expression of the nitric oxide synthase from said nucleic acid molecule is regulated by a non-constitutive promoter.

9. The method of claim 1, wherein expression of said nitric oxide synthase is regulated by a promoter which is specifically active in cholinergic ganglia tissue.

10. The method of claim 1 wherein said nucleic acid molecule is packaged within a non-viral gene therapy vector.

11. The method of claim 10, wherein said nucleic acid molecule is delivered as naked DNA.

12. The method of claim 3, wherein cardiac vagal tone in the patient is increased by at least 10%.

13. The method of claim 1, wherein said nucleic acid molecule comprises DNA.

14. The method of claim 1, wherein said nucleic acid molecule is non-replicating.

15. The method of claim 1, wherein said nucleic acid molecule is non-integrating.

16. The method of claim 1, wherein said nucleic acid molecule is an autonomously replicating episomal or extrachromosomal vector, such as a plasmid.

17. The method of claim 1, wherein said nucleic acid molecule is delivered to the heart within microbubbles which can be disrupted by ultrasound.

18. A nucleic acid molecule comprising a non-constitutive promoter and a coding sequence, wherein: (a) said promoter is operably linked to the coding sequence to control transcription of the coding sequence; (b) said promoter is specifically active in cholinergic ganglia tissue; and (c) said coding sequence encodes a nitric oxide synthase.

19. A pharmaceutical composition comprising the nucleic acid molecule of claim 18.
